(19) United States
(12) Patent Application Publication
(10) Pub. No.: US 2015/0202397 A1
(43) Pub. Date: Jul. 23, 2015

(54) PATIENT INTERFACE ASSEMBLY WITH FORCE LIMITER

(71) Applicant: KONINKLIJKE PHILIPS N.V., EINDHOVEN (NL)

(72) Inventors: Sander Theodoor Pastoor, Eindhoven (NL); Peter Chi Fai Ho, Pittsburgh, PA (US); Harmina Christina Zeijlstra, Eindhoven (NL); Krijn Frederik Bustraan, Eindhoven (NL)

(21) Appl. No.: 14/418,492

(22) PCT Filed: Jul. 12, 2013
(86) PCT No.: PCT/IB2013/055733

§ 371 Date: Jan. 30, 2015

Related U.S. Application Data
(60) Provisional application No. 61/679,105, filed on Aug. 3, 2012.

(51) Int. Cl. A61M 16/06; A61M 16/08

(52) U.S. Cl. CPC A61M 16/0622; A61M 16/0633; A61M 16/0683; A61M 16/08

(57) ABSTRACT

The present invention relates to a patient interface assembly (10) and a force limiter (40). The force limiter (40) is adapted to limit a force between a patient interface (20) and a patient's face (14) when the patient interface (20) is applied to the patient (14). For this purpose, the force limiter (40) comprises a spring-like element (47, 48, 49, 49′, 49″, 49″, 1, 70) with a substantially degressive spring characteristic. The present invention further 5 relates to a patient interface, an attachment assembly and a clip for use in a patient interface assembly and comprising said force limiter.

### FIELD OF THE INVENTION

The present invention relates to a patient interface assembly with a force limiter for preventing to overtighten a patient interface on a patient's face and a force limiter for use in a patient interface assembly.

### BACKGROUND OF THE INVENTION

Patient interfaces, such as masks for covering the mouth and/or nose, are used for delivering gas to a patient. Such gases, like air, cleaned air, oxygen, or any modification of the latter, are submitted to the patient via the patient interface in a pressurized or unpressurized way.

For several chronic disorders and diseases, a long-term attachment of such a patient interface to a patient is necessary or at least advisable.

One non-limiting example for such a disease is obstructive sleep apnea or obstructive sleep apnea syndrome (OSA). OSA is usually caused by an obstruction of the upper airway. It is characterized by repetitive pauses in breathing during sleep and is usually associated with a reduction in blood oxygen saturation. These pauses in breathing, called apneas, typically last 20 to 40 seconds. The obstruction of the upper airway is usually caused by reduced muscle tonus of the body that occurs during sleep. The human airway is composed of walls of soft tissue which can collapse and thereby obstruct breathing during sleep. Tongue tissue moves towards the back of the throat during sleep and thereby blocks the air passages. OSA is therefore commonly accompanied with snoring.

Different invasive and non-invasive treatments for OSA are known. One of the most powerful non-invasive treatments is the usage of Continuous Positive Airway Pressure (CPAP) or Bi-Positive Airway Pressure (BiPAP) in which a patient interface, e.g. a face mask, is attached to a tube and a machine that blows pressurized gas, preferably air, into the patient interface and through the airway in order to keep it open. Positive air pressure is thus provided to a patient through a hose connected to a patient interface or respiratory interface, such as a face mask, that is worn by the patient. The aforementioned long-term use of the patient interface is the result, since the wearing of the patient interface takes place during the sleeping time of the patient.

Examples for patient interfaces are:

nasal masks, which fit over the nose and deliver gas through the nasal passages,

oral masks, which fit over the mouth and deliver gas through the mouth,

full face masks, which fit over both, the nose and the mouth, and deliver gas to both, and

nasal pillows, which are regarded as masks as well within the scope of the present invention and which consist of small nasal inserts that deliver the gas directly to the nasal passages.

The patient interface is usually positioned on the patient's head using some kind of headgear. The combination of patient interface and headgear is referred to as patient interface assembly. Wearing a patient interface can be uncomfortable, since for providing an airtight seal between the patient interface and the patient's face, the patient interface has to be worn tightly on the face. Further, the patient interface may comprise a forehead support. Such a forehead support is often designed as a pad that touches the forehead of a patient during use. The offset of the forehead support in direction to the user's forehead allows an adjustment of the angle of the mask on the user's face. A forehead support is often included in order to relieve the pressure of the patient interface on the nose bridge.

In many products, for example the Philips Respironics EasyLife CPAP or ResMed Mirage FX masks, the position of the mask and forehead offset are controlled by the strap force. There is a risk of overtightening the straps to find a personal leak-free setting.

In other products, for example the ResMed MicroFit™ dial solutions, a separate component is provided for an offset adjustment of the forehead support. This additional component decouples the offset of the forehead support from the strap force. However, solutions with additional components can be bulky and relatively rigid, which can create pressure points in non-ideal positions.

Apart from being uncomfortable, the tightly wearing of the patient interface on the face may result in red marks once the patient interface is removed.

### SUMMARY OF THE INVENTION

It is an object of the present invention to provide a patient interface assembly providing increased user comfort and a reduction of the formation of pressure marks, preferably avoiding them completely, and which therefore improves the comfort for a patient wearing a patient interface.

In a first aspect of the present invention, a patient interface assembly is presented that comprises a patient interface for delivering a flow of breathable gas to a patient, an attachment assembly for attaching the patient interface to a patient's face, and a force limiter for limiting a force between the patient interface and a patient's face when the patient interface is applied to the patient by limiting a force of the attachment assembly on the patient interface. Further, the force limiter comprises a spring-like element with a substantially degressive spring characteristic.

In a further aspect of the present invention, a force limiter for use in a patient interface assembly is presented. The force limiter is adapted to interact with an attachment assembly for attaching the patient interface to a patient's face. Further, the force limiter is adapted to limit a force between the patient interface and the patient's face when the patient interface is supplied to the patient by limiting a force of the attachment assembly on the patient interface. For this purpose, the force limiter comprises a spring-like element with a substantially degressive spring characteristic.

In a further aspect of the present invention, a patient interface for use in a patient interface assembly as described above is presented, wherein the patient interface comprises the aforementioned force limiter.

In a further aspect of the present invention, an attachment assembly for use in a patient interface assembly as described above is presented, wherein the attachment assembly comprises the aforementioned force limiter.

In a further aspect of the present invention, a clip for use in an attachment assembly is presented, wherein the clip is adapted to attach the attachment assembly to a patient interface in a patient interface assembly as described above, wherein the clip comprises the aforementioned force limiter.

Preferred embodiments of the invention are defined in the dependent claims. It shall be understood that the claimed force limiter has similar and/or identical preferred embodiments as the claimed patient interface assembly and as defined in the dependent claims.

With the aid of the aforementioned force limiter, it is possible to limit the force with which the patient interface is pressed onto the patient's face. A disadvantage of most existing adjustment mechanisms is the fact that the user of a patient interface assembly is adjusting the force that the patient interface exerts on the face by him or herself. Being generally not an expert in this matter, the patient interface is often either put on with too high a force or too low a force. Too high a force may easily cause discomfort, leave visually very distractive red pressure marks or even cause skin damage and significant pain. Too low a force may cause air leaks, defeating the purpose of the pressure support system, and cause a skin discomfort as well as waking-up sleeping patients due to unpleasant noise. Additionally, the air can also leak into the patient's eyes and cause eye irritation. In order to ensure that the gas is actually delivered, patients tend to overtighten the patient interface when attaching the patient interface assembly.

The force limiter according to the present invention is adapted to interact with the attachment assembly, in particular by limiting the force that the attachment assembly exerts on the patient interface when attaching the patient interface to a patient's face. For this purpose, the force limiter comprises a spring-like element with a substantially degressive spring characteristic. The term “degressive spring characteristic” as used within the context of the present invention is to be understood as a spring characteristic wherein upon approaching a threshold force, the spring constant of the spring-like element reduces.

According to an embodiment of the present invention, upon reaching a threshold force, the spring constant of the spring-like element is substantially zero. Thereby, the spring-like element elongates when the straps of the attachment assembly are tightened further, however, the force exerted on the patient interface does not further increase by the same amount. The term “spring constant” within the scope of the present invention also refers to the instantaneous spring constant or derivative of the force-strain curve. Hence, when the spring constant is substantially zero this can also refer to the derivative of the force-strain curve being zero or close to zero. In other words, upon reaching a threshold force, the spring-like element further elongates, however, this elongation does not increase the restoring force of the spring-like element that presses the patient interface on the patient's face. In other words, the spring-like element allows the attachment strap to elongate slowly to absorb the tension without tightening (in physical way shortening the straps) the patient interface no matter how much force is reasonably applied by the users (normally around 2 to 5 lbf or 9 to 22 Newton).

According to another embodiment of the patient interface assembly, the spring-like element is formed by a bellow structure. This structure acts as a spring bellow that exerts a restoring force when it is elongated.

According to an alternative embodiment, the spring-like element is formed by an elastic material. Generally, the spring-like element is not limited to a particular form. Any structure adapted to provide a substantially degressive spring characteristic can be employed, including metal springs, bellow structures, elastic materials, gas springs or combinations of springs and damping elements. In particular a spring characteristic, wherein upon reaching a threshold force, the spring constant or derivative of the spring constant reduces favorably to zero, is well suited for the given application. It goes without mentioning that the spring-like element exhibits the desired spring characteristic over a limited range only, as any spring element will ultimately reach a damage threshold.

According to an embodiment of the patient interface assembly, the attachment assembly comprises a clip and wherein said clip comprises the force limiter. The clip within the scope of the present invention is an element adapted to establish a connection between the patient interface and the attachment assembly. The patient interface can be adapted to provide means for the clip to engage with.

According to an alternative embodiment, the attachment assembly comprises a strap and said strap comprises the force limiter. The force limiter can also be glued or stitched against said strap.

According to another embodiment of the patient interface assembly, the patient interface comprises a mask shell and said mask shell comprises the force limiter. The force limiter can thus be formed as an integral part of the patient interface, in particular of the mask shell. Force limiter and mask shell can be made of materials with same or different elasticity.

According to an alternative embodiment, the patient interface comprises a cushion further comprising an extension flap extending from said cushion adapted as the force limiter. Hence, the extension flap acts as the force limiter.

The term “sealing cushion” or “cushion” as used within the context of the present invention is to be understood a part of the patient interface normally comprising a soft material and being arranged on the side of the patient interface that is directed to and is getting into contact with the patient when the patient interface is worn by the patient. Thereby the cushion provides an airtight seal and/or makes the wearing of the patient interface more comfortable for the patient. Therefore, the cushion may be a part of the mask part which provides the gas to the patient or any other part of the patient interface that gets into contact with the patient, like a forehead support. Accordingly, other words for describing this “sealing cushion” would be cushion, seal, pad or the like.

According to another embodiment of the patient interface assembly, the patient interface comprises a mask shell and the mask shell is adapted to guide the extension flap of the cushion. For example, the mask shell is adapted to support the cushion. The mask shell may further comprise a hole through which the extension flap fits. Alternatively, the extension flap can be guided by ridges formed in the mask shell.

According to another embodiment of the present invention, the force limiter is made of at least a material of a group of materials comprising 2 k mold, silicone and rubber. Preferably at least the spring-like element is made from an elastic material. Fabrication techniques for producing the force limiter include but are not limited to injection molding or two component injection molding.

According to another embodiment of the present invention, the patient interface assembly further comprises a plurality of force limiters being arranged at different locations of the patient interface assembly. For example two force limiters can be arranged on a mask shell of the patient interface, a first force limiter at the left side and a second force limiter at the right side. Alternatively, four force limiters can be arranged at the mask shell, two on the left side, two on the right side. Alternatively, at least one force limiter is arranged adjacent to the patient interface, whereas another force limiter is located elsewhere on the attachment assembly. Alternatively, at least one force limiter is arranged adjacent to a forehead support of the patient interface. Still further, a force limiter can be arranged in or on the attachment assembly, for example on the back of the patient's head.

According to another embodiment of the present invention, the force limiter comprises a plurality of spring-like elements each with its own spring characteristic, wherein the combination of the spring-like elements exhibits a substantially degressive spring characteristic. In other words, the desired spring characteristic can be a composition of multiple spring elements. For example, the force limiter comprises two bellow structures with different spring characteristics. Alternatively, the force limiter is made of a plurality of different elastic materials. In a further alternative, any combination of metal springs, gas springs, bellow structures and elastic materials as well as damping elements is used for designing the force-limiter.

### BRIEF DESCRIPTION OF THE DRAWINGS

These and other aspects of the invention will be apparent from and elucidated with reference to the embodiments described hereinafter. In the following drawings

FIG. 1 shows a schematic perspective view of a patient wearing a patient interface assembly according to the present invention,

FIG. 2 shows a force-strain diagram using the force limiter according to the present invention,

FIG. 3 shows an attachment assembly connected to a patient interface by a clip comprising the force limiter according to the present invention,

FIG. 4A shows a first embodiment of a clip comprising the force limiter according to the present invention,

FIG. 4B shows a second embodiment of a clip comprising the force limiter according to the present invention,

FIG. 5A shows a first embodiment of a strap comprising the force limiter according the present invention,

FIG. 5B shows a second embodiment of a strap comprising the force limiter according to the present invention,

FIG. 5C shows a third embodiment of a strap comprising the force limiter according to the present invention,

FIG. 6 shows an embodiment of a mask shell comprising the force limiter according to the present invention,

FIG. 7A shows a first embodiment of a cushion comprising an extension flap adapted as a force limiter, and

FIG. 7B shows a second embodiment of a cushion comprising an extension flap adapted as a force limiter wherein the mask shell guides the extension flap.

### DETAILED DESCRIPTION OF THE INVENTION

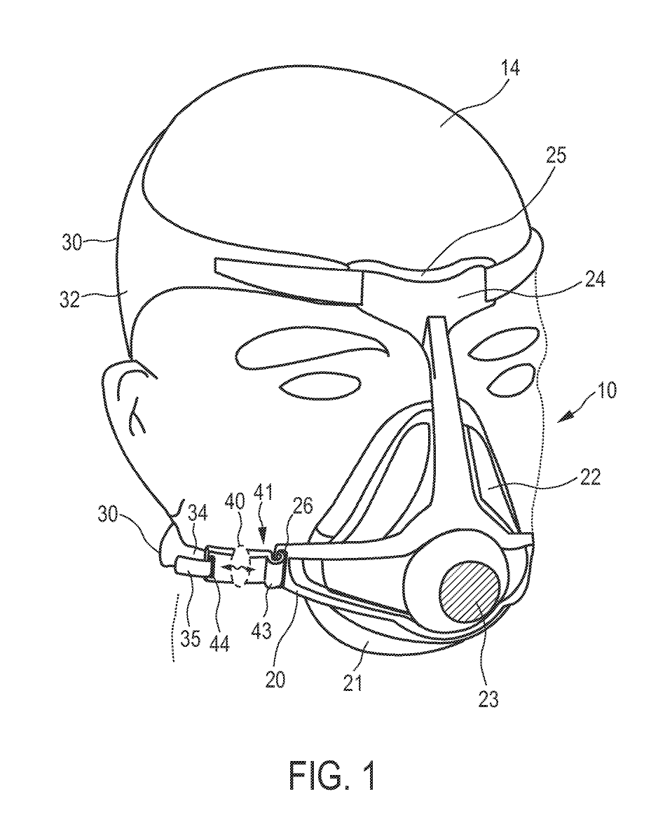

An embodiment of a patient interface assembly according to the present invention is shown in FIG. 1 and is designated in its entirety by the reference numeral 10. The patient interface assembly 10 is worn by a patient 14. In this particular embodiment, the patient interface assembly 10 comprises a patient interface 20 in form of a full face mask covering the mouth and nose of the patient 14. The full face mask 20 comprises a sealing cushion 21 and a shell 22. The cushion 21 is arranged on the shell 22 on that side that is directed to the face of the patient 14 in order to make the wearing of the full face mask 20, and of the patient interface assembly 10 in general, more comfortable and especially to provide an airtight seal of the full face mask 20 on the patient's face 14. For this, the cushion 21 is comprised of a soft material, like silicone rubber or any other rubber or suitable elastic material. On the opposite side directing away from the patient's face, the shell 22 comprises a connector 23. Via this connector 23 the patient interface 20 is able to be connected to a hose (not shown) via which the (pressurized) gas can be submitted to the patient 14. In order to reduce the pressure on the nose bridge of the patient 14 the patient interface further comprises a forehead support 24. This forehead support 24 is directly connected to the shell 22 of the full face mask 20. Thereby, a more even distribution of the force exerted on the nose region of the patient 14 is achieved. For improving the comfort when the forehead support 24 is pressing against the skin of the patient 14, the forehead support 24 comprises a forehead cushion 25.

For attaching the patient interface 20 on the patient 14, the patient interface assembly 10 further comprises an attachment assembly 30 in form of a headgear. This headgear 30 is in this particular embodiment of FIG. 1 comprised of two straps 32 and 34 circumventing the head of the patient 14, thereby attaching the patient interface 20 with a certain attachment force on the patient's face 14.

Because of this attachment force, the patient interface 20, especially the sealing cushion 21 presses against the skin of the patient 14 that lies under said cushion 21. This may result in the formation of red marks due to the stop of the blood flow in the arteries within the skin of the patient 14 in this area. Also, the forehead cushion 25 of the forehead support 24 presses against the skin of the patient 14, which may also lead to the formation of red marks in that area.

The patient interface assembly 10 shown in FIG. 1 further comprises a clip element 41. The clip 41 connects the strap 34 of the attachment assembly 30 with the mask shell 22 of the patient interface 20. For this purpose, the particular embodiment of a clip 41 shown here features a strap slot 44 for connection with the head gear strap 34. On the other side, the clip 41 features a hook 43 that is adapted to engage with an opening 26 in the shell 22 of the patient interface 20.

By pulling the strap end 35 of the head gear strap 34, the length of the head gear strap 34 that circumvents the head of the patient 14 is reduced. This increases the force with which the patient interface 20 is pressed on the patient's face 14. This strapping force increases with the displacement or strain of the strap end 35. However, there is no need to overtighten the strap. Once an airtight seal has been reached, there is no need to further increase the pressure of the patient interface onto the patient's face 14. Hence, the clip 41 further comprises a force limiter 40 according to the present invention having a spring-like element with a substantially degressive spring characteristic. When pulling the end 35 of the head gear strap 34, the force exerted on the patient interface 20 increases. Due to the force limiter 40, the strap force will be limited to a threshold value which is in the correct range not to overtighten the patient interface 20 with the cushion 21 on the patient's face 14. Hence, the force is sufficiently strong to ensure an air-tight seal on the patient's face but does not cause the formation of pressure marks.

According to another embodiment of the present invention, force limiters with different threshold values or tunable threshold values can be selected based on the threshold value that is required for a particular patient. For example, a child will typically require a lower force for tightening the patient interface 20 than an adult.

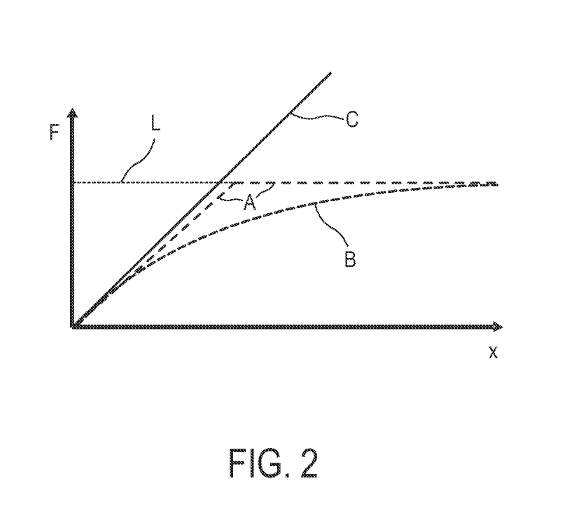

FIG. 2 shows a diagram of the strapping force or strapping tension on the y-axis over the strap displacement or strain on the x-axis. The spring constant is given as the ratio of the strapping force or restoring force F held by said strap over the strap displacement x. Curve C shows a linear relationship between strapping force F and strap displacement x corresponding to a linear spring characteristic. In other words, the higher the strap displacement the higher the strapping force. The derivative of the spring constant remains constant over the strap displacement

$$\frac{Δ F}{Δ x} = const .$$

Such a relationship between strap displacement and strapping force allows the patient 14 to overtighten the patient interface 20 and compressing the cushion 21 more than necessary.

Curves A and B in FIG. 2 show the relationship between strap displacement and strapping force of a force limiter 40 according to the present invention. In particular, the strapping force is limited by a threshold value L that prevents overtightening the patient interface 20 on the patient's face 14.

Curve A in FIG. 2 shows an idealized relationship between strap displacement and strapping force. The strapping force increases with increased strap displacement until reaching the threshold value L. Before reaching the threshold value L, the derivative of the spring constant is a constant value

$$\frac{Δ F}{Δ x} = const .$$

At higher strap displacement, having reached the threshold value L, the derivative of the spring constant of an idealized force limiter 40 is

$$\frac{Δ F}{Δ x} = 0.$$

Curve B in FIG. 2 shows an alternative graph of the relationship between strap displacement and strapping force for a force limiter 40 having a spring-like element with a substantially degressive spring characteristic. The spring constant is not a constant value but decreases with increasing strap displacement. Hence, upon approaching a threshold force L, the derivative of the spring constant of the spring-like element reduces further and further. In an alternative embodiment of the force limiter, the threshold force L is not a fixed threshold but rather indicates a value at which the derivative of the spring constant of the spring-like element has reduced close to zero. In other words, when getting close to the threshold force, further pulling the strap end 35 does not substantially increase the strapping force any further. Hence, the force limiter 40 according to the present invention prevents the patient 14 from overtightening the patient interface 20 on his/her face.

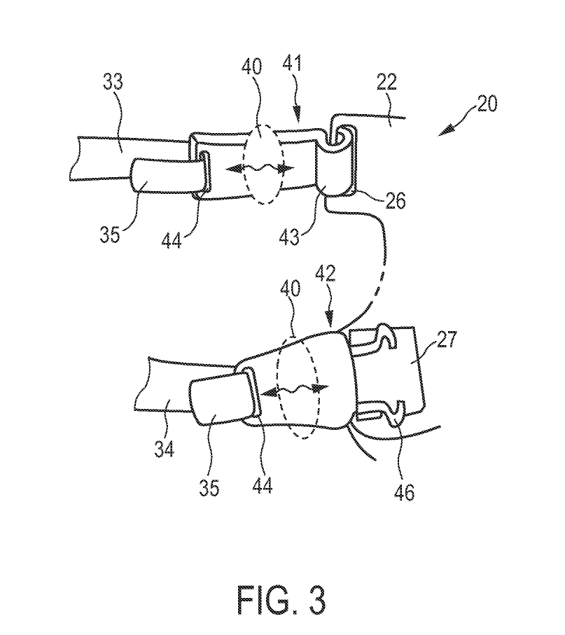

FIG. 1 has shown a patient interface 20 with a shell 22 that is attached to a strap 34 of the attachment assembly 30 with one clip element 41. FIG. 3 shows an alternative embodiment of a patient interface 20 with a shell 22 that is attached to an attachment assembly 30 having a first head gear strap 33 and a second head gear strap 34. At one end, the clip 41 has a strap slot 44 where the head gear strap 33 is fastened. Preferentially, a type of fastening is used that enables convenient strap displacement. For example a Velcro strap or the like can be used. The clip 41 further comprises a force limiter 40 with a strap force-strain-relationship as previously explained in FIG. 2. The clip 41 features a hook 43 to engage with an opening 26 in the shell 22 of the patient interface 20.

According to an alternative embodiment, the second head gear strap 34 can be attached to the shell 22 of the patient interface 20 via quick-release-clip 46 and the corresponding snap-cup 27 in the shell 22.

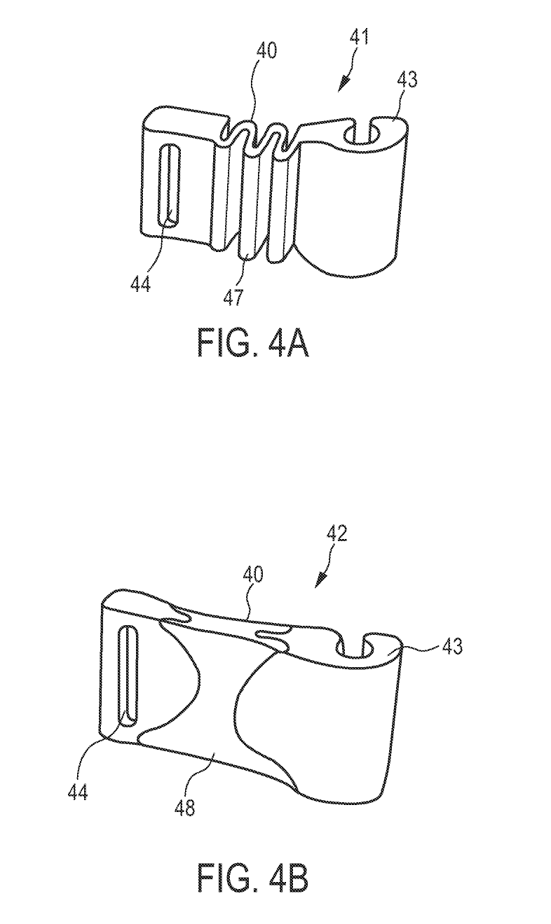

FIGS. 4A and 4B show two alternative embodiments of a spring-like element according to the present invention. FIG. 4A shows a clip 41 with a strap slot 44 for connection with the attachment assembly 30 on the one side, a hook 43 for engaging with the patient interface 20 on the other side and a force limiter 40 in between. It should be noted that the clip 43 is not limited to this particular form but can be any type of connection with the patient interface 20. In this particular embodiment, the spring-like element of the force limiter 40 is implemented as a bellow flexible structure 47. This structure 47 allows flexible elongation of the force limiter 40 and is adapted to follow a spring characteristic as previously shown in FIG. 2 curves A or B.

In an alternative embodiment, the spring-like element comprises a plurality of springs to achieve the overall desired spring characteristic. Such a spring-like element that benefits from different spring characteristics of its sub-elements can also be implemented with a rubbery structure using a plurality of different materials.

FIG. 4B shows an alternative embodiment of a clip element 42. The first end of the clip 42 comprising the strap slot 44 is made of a first material, the force limiter 40 is made of a second material and the side comprising the hook is again made of the first material. In other words, the clip is fabricated using two materials, in particular two materials with different elasticity. The spring-like element 48 of the force limiter 40 is implemented using an elastic material such as silicone or rubber. The spring like element 48 is designed for a force-strain curve as shown in FIG. 2 curves A or B. Preferably, the clip element 42 can be produced in a 2 k injection molding process for low cost manufacturing.

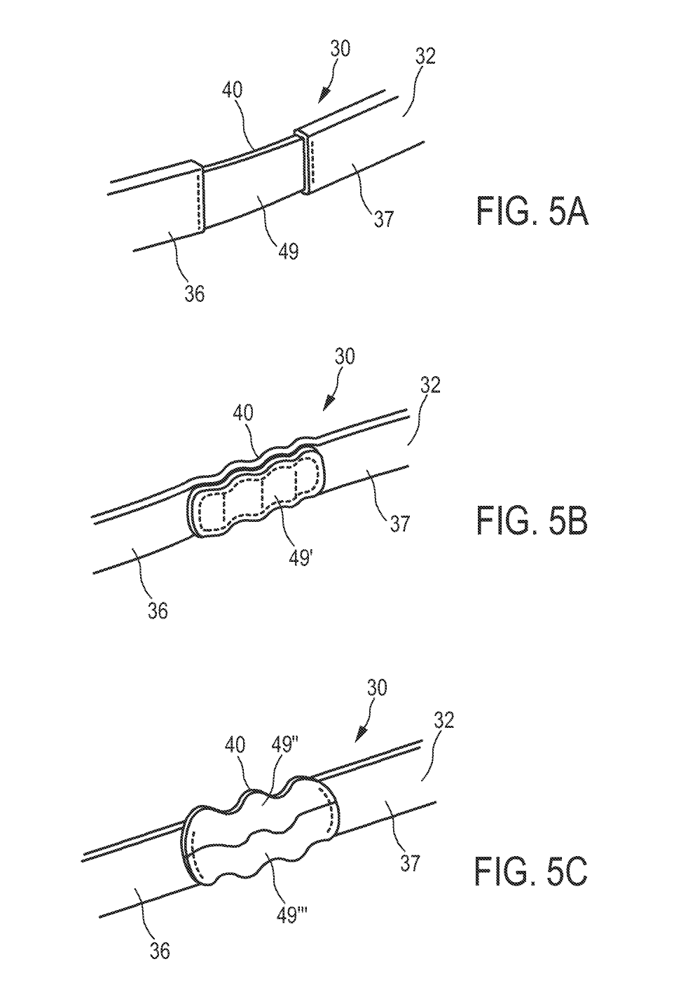

FIGS. 5A, 5B and 5C show alternative embodiments of an attachment assembly with a strap 32 having the force limiter 40 integrated into or attached to the strap. FIG. 5A shows a first embodiment for a strap 32 having a first side 36 and a second side 37 with a force limiter 40 in between. In this particular embodiment, the force limiter 40 with its spring-like element 49 is implemented as a special section of the strap 32 having a reduced strap stiffness by locally adding more flexibility. This can also be done with locally reduced strap thickness or a cross-section of an appropriate material with the desired elasticity. FIG. 5A shows the force limiter under tension. Some of the strapping force is absorbed by the device.

As shown in FIG. 5B, a lower stiffness can also be reached by stitching or locally gluing a flexible bending bellow-like element 49′ on the strap that acts as the spring-like element of a force limiter 40. FIG. 5B shows the force limiter without tension. The bellow structure 49′ ensures that the force is exerted on the force limiter 40 and not the strap 33 in the portion where the bellowed element 49′ is attached to the head gear strap 33. For example, the headgear strap is an elastic or non-elastic flexible band that follows the curves of the bellow structure 49′ without limiting a straightening of the bellow structure 49′ such that the bellow structure can exert its restoring force.

In FIG. 5C, a flexible bending bellow-like element is inserted between a first end 36 and a second end 37 of a head gear strap 32 of the attachment assembly 30. Again, this element acts as a spring-like element of the force limiter 40 with a force-strain curve according to curves A or B in FIG. 2. In this embodiment, the upper half 49″ of the bellow-like element is made from a first material with a first spring characteristic and the lower half 49′ of the element from a second material with a second spring characteristic. The combination of both sub-elements provides the desired behavior. Alternatively, a combination with further materials or different ratios such as an upper third of a first material and two lower thirds of a second material are possible.

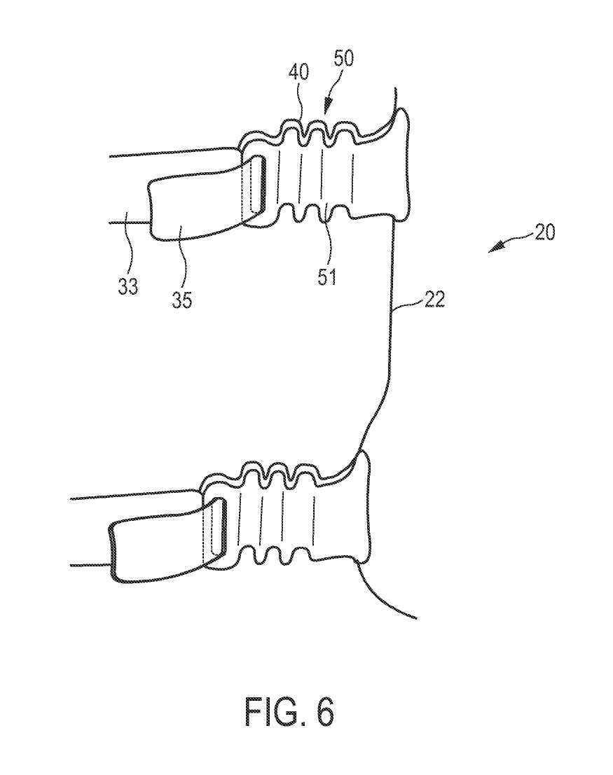

The embodiment shown in FIG. 6 discloses a patient interface 20 having a mask shell 22 wherein the force limiter 40 is formed as an extension part 50 of the mask shell 22. The spring-like element is implemented as a flexible bellow structure 51 in the mask shell 22. The head gear strap 33 is guided through a strap slot 54 such that a loose end 35 can be used to tighten the strap and thereby to increase the strapping force. In a preferred embodiment, the mask shell 22 and the extension 50 are made from different materials, for example the mask shell 22 is made from a rather rigid material whereas the extension 50 is made from an elastic material such as silicone or rubber. Fabrication techniques include 2 k injection molding that can be used to integrate a less elastic material for the mask shell 22 and a more elastic material for the spring-like element 51.

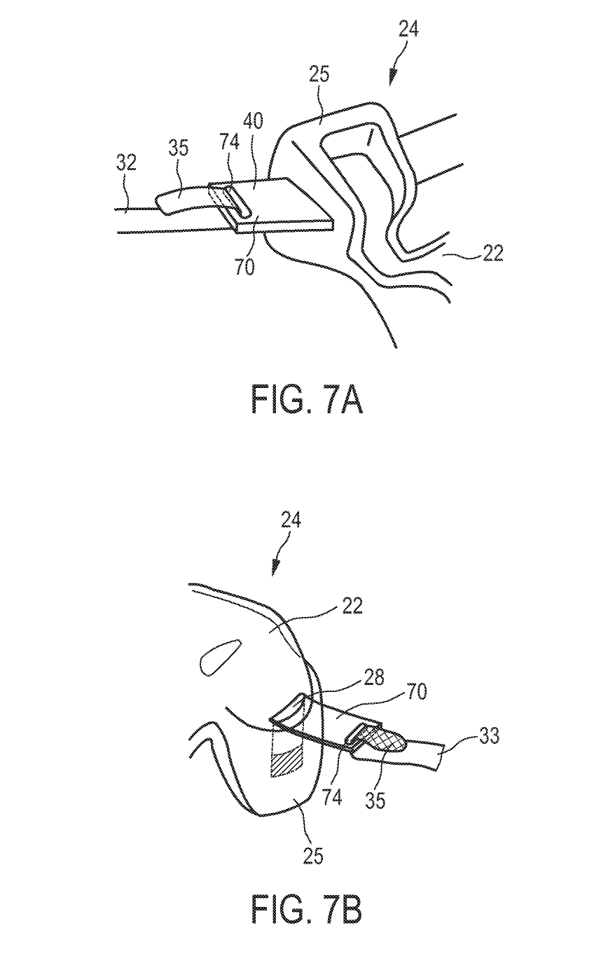

In yet another embodiment, as shown in FIGS. 7A and 7B, the force limiter 40 is attached to the forehead support 24. FIG. 7A shows a perspective view of the forehead support part 24 of a patient interface 20 from the side facing the patient's face 14. The forehead support 24 comprises a forehead cushion 25 made from an elastic flexible material. The forehead cushion further comprises an extension flap 70 that acts as the spring-like element. The extension flap 70 further features a strap slot 74 through which the head gear strap 32 can be guided such that the attachment assembly can be fastened by pulling the loose end 35 of the head gear strap 32. In FIG. 7A, the extension flap 70 is directly connected to the forehead cushion 25. Alternatively, the extension flap 70 can be guided through a hole 28 in the mask shell 22 of the forehead support 24 as shown in FIG. 7B.

In conclusion, the patient interface assembly and force limiter according to the present invention effectively mitigate the problem of overtightening a patient interface 20 and thereby increase patient comfort. This is achieved by a force limiter with a variable spring constant or spring rate. The spring-like element modifies the loading to reduce or to limit the strapping tension to avoid overtightening the patient interface on the patient's face. In particular, the force limiter can be easily implemented within clip elements 41, 42, straps 32, 33, 34 of the attachment assembly or co-integrated with already existing elements such as the mask shell 22 or cushions 21, 25 to allow cost-efficient manufacturing.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A patient interface assembly comprising:
a patient interface for delivering a flow of breathable gas to a patient,
an attachment assembly for attaching the patient interface to a patient's face, and
a force limiter for limiting a force between the patient interface and the patient's face when the patient interface is applied to the patient by limiting a force of the attachment assembly on the patient interface, and wherein the force limiter comprises a spring-like element with a substantially degressive spring characteristic.

2. The patient interface assembly according to claim 1, wherein, upon reaching a threshold force, the spring constant of the spring-like element is substantially zero.

3. The patient interface assembly according to claim 1, wherein the spring-like element is formed by a bellow structure.

4. The patient interface assembly according to claim 1, wherein the spring-like element is formed by an elastic material.

5. The patient interface assembly according to claim 1, wherein the attachment assembly comprises a clip and wherein said clip comprises the force limiter.

6. The patient interface assembly according to claim 1, wherein the patient interface comprises a mask shell and wherein said mask shell comprises the force limiter.

7. The patient interface assembly according to claim 1, wherein the patient interface comprises a cushion further comprising an extension flap extending from said cushion adapted as the force limiter.

8. The patient interface assembly according to claim 7, wherein the patient interface comprises a mask shell and wherein mask shell is adapted to guide the extension flap of the cushion.

9. The patient interface assembly according to claim 1, wherein the force limiter is made of at least a material of a group of materials comprising 2 k mold, silicone and rubber.

10. The patient interface assembly according to claim 1, further comprising a plurality of force limiters being arranged at different locations of the patient interface assembly.

11. The patient interface assembly according to claim 1, wherein the force limiter comprises a plurality of spring-like elements each with its own spring characteristic, wherein the combination of the spring-like elements exhibits a substantially degressive spring characteristic.

12. A force limiter for use in a patient interface assembly according to claim 1, wherein the force limiter is adapted to limit a force between the patient interface and the patient's face when the patient interface is applied to the patient by limiting a force of the attachment assembly on the patient interface, the force limiter comprising a spring-like element with a substantially degressive spring characteristic.

13. A patient interface for use in a patient interface assembly according to claim 1, wherein the patient interface comprises a force limiter.

14. An attachment assembly for use in a patient interface assembly according to claim 1, wherein the attachment assembly comprises a force limiter.

15. A clip for use in an attachment assembly wherein the clip is adapted to attach the attachment assembly to a patient interface in a patient interface assembly according to claim 1, wherein the clip comprises a force limiter.
